(19) United States
(12) Patent Application Publication
(10) Pub. No.: US 2010/0087731 A1
(43) Pub. Date: Apr. 8, 2010

(54) Method for Tracking Degradation of a Biodegradable Stent Having Superparamagnetic Iron Oxide Particles Embedded Therein

(75) Inventor: Meena Ramachandran, San Francisco, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/246,752

(22) Filed: Oct. 7, 2008

(51) Int. Cl. A61B 5/05; A61B 19/00; A61F 2/06

(52) U.S. Cl. 600/424; 128/898; 623/1.49

(57) ABSTRACT

A tubular stent formed from a plurality of filaments, the filaments constructed out of a solid bioabsorbable polymeric material having active agent particles dispersed there through that are visible by magnetic resonance imaging (MRI). The active agent particles are superparamagnetic iron oxide (SPIO) particles. The SPIO particles enhance the visibility of the polymeric stent under MRI, and also allow for accurate monitoring of stent degradation. As the stent degrades, the SPIO particles are released and either flow downstream or are embedded by nearby macrophages. The amount of SPIO particles within the remaining stent body is decreased, which results in a different MRI signal. By quantifying the signal change, the amount of biodegradable stent remaining can be deduced in situ and the stent degradation rate may be accurately calculated.

### FIELD OF THE INVENTION

The invention relates generally to temporary endoluminal prostheses for placement in a body lumen, and more particularly to stents that are biodegradable.

### BACKGROUND OF THE INVENTION

A wide range of medical treatments exists that utilize “endoluminal prostheses.” As used herein, an endoluminal prosthesis is intended to cover a medical device that is adapted for temporary or permanent implantation within a body lumen, including both naturally occurring and artificially made lumens, such as without limitation: arteries, whether located within the coronary, mesentery, peripheral, or cerebral vasculature; veins; gastrointestinal tract; biliary tract; urethra; trachea; hepatic shunts; and fallopian tubes.

Accordingly, a wide assortment of endoluminal prostheses have been developed, each providing a uniquely beneficial structure to modify the mechanics of the targeted lumen wall. For example, stent prostheses are known for implantation within body lumens to provide artificial radial support to the wall tissue, which forms the various lumens within the body, and often more specifically, for implantation within the blood vessels of the body.

Essentially, stents are made to be permanently or temporarily implanted. A permanent stent is designed to be maintained in a body lumen for an indeterminate amount of time and is typically designed to provide long-term support for damaged or traumatized wall tissues of the lumen or to maintain the patency of a vessel clogged with atherosclerotic plaque. There are numerous conventional applications for permanent stents including cardiovascular, urological, gastrointestinal, and gynecological applications. A temporary stent is designed to be maintained in a body lumen for a limited period of time in order to maintain the patency of the body lumen, for example, after trauma to a lumen caused by a surgical procedure or an injury or to temporarily open a clogged lumen until natural healing occurs.

Permanent stents, over time, may cause irritation to the surrounding tissue resulting in inflammation at the implant site and restenosis, or re-narrowing of the vessel lumen. Further, if an additional interventional procedure is ever warranted, a previously permanently implanted stent may make it more difficult to perform the subsequent procedure.

Temporary stents, on the other hand, avoid the complications associated with long-term implants. Temporary stents may advantageously be eliminated from body lumens after an appropriate period of time, for example, after the traumatized tissues of the lumen have healed and a stent is no longer needed to maintain the patency of the lumen. As such, temporary stents may be removed surgically or be made bioabsorbable/biodegradable.

Temporary stents may be made from bioabsorbable and/or biodegradable materials that are selected to absorb or degrade in vivo over time. However, there are considerations associated with the use of bioabsorbable or biodegradable stents, such as how to control the breakdown of the bioabsorbable materials from which such stents are made, as in, preventing the material from breaking down too quickly or too slowly. If the material is absorbed too quickly, the stent will not provide sufficient time for the vessel to heal, or if absorbed too slowly, the attendant disadvantages of permanently implanted stents may arise.

The use of biodegradable polymeric stents presents another challenge associated with visualizing the stent post-implantation. Magnetic resonance imaging (MRI) offers several potential advantages for post-implantation evaluation of the biodegradable stent, including the potential for mitigating radiation and contrast-related side effects. In addition, MRI-guided stent evaluation is non-invasive and may offer image acquisition in any desired orientation and three-dimensional (3D) soft-tissue contrast with simultaneous visualization of the interventional device. Thus, it is desirable to provide a biodegradable polymeric stent that is visible or detectable under magnetic resonance imaging.

In addition, since biodegradable polymeric stents inherently lack radiopacity and are only visible with highly invasive intravascular ultrasound, it is difficult to assess stent degradation during follow-up procedures. Thus, there remains a need to non-invasively monitor the degradation of the biodegradable polymeric stent in situ in order to accurately quantify the stent degradation rate.

### BRIEF SUMMARY OF THE INVENTION

A method of non-invasively tracking the degradation of a biodegradable stent implanted in a body lumen is disclosed. A stent of a biodegradable polymeric material with superparamagnetic iron oxide particles embedded therein is provided and positioned within the body lumen. A first image of the stent is obtained by magnetic resonance imaging at a first time upon implantation. A second image of the stent is obtained by magnetic resonance imaging using the same pulse sequence and imaging parameters as used in obtaining the first image at a second time, the second time occurring after the first time. The first image and the second image are compared to determine the amount of stent remaining in the body lumen. In addition, the degradation rate of the stent may be calculated by quantifying the amount of stent remaining in the body lumen and the amount of stent that has degraded over the time period that has passed between the first time and the second time.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of an exemplary stent formed of a plurality of braided filaments, wherein the stent is in an expanded configuration.

FIG. 1A is a cross-sectional view of a stent filament taken along line A-A of FIG. 1.

FIG. 2 is an end view of the stent in FIG. 1.

FIG. 3 is a side view of the stent of FIG. 1, wherein the stent is in a contracted configuration.

FIG. 4 is an end view of the stent in FIG. 3.

FIG. 5 is a side view of an exemplary stent delivery system.

FIG. 6 illustrates the steps of tracking the degradation of a biodegradable stent implanted in a body lumen.

### DETAILED DESCRIPTION OF THE INVENTION

Specific embodiments are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. More particularly, the stents are adapted for deployment at various treatment sites within the patient, and include vascular stents (e.g., coronary vascular stents and peripheral vascular stents such as cerebral stents), urinary stents (e.g., urethral stents and ureteral stents), biliary stents, tracheal stents, gastrointestinal stents and esophageal stents. Furthermore, there is no intention to be bound by any expressed or implied theory presented in the preceding technical field, background, brief summary or the following detailed description.

Embodiments hereof are directed to biodegradable stents that are visible to a physician under magnetic resonance imaging (MRI) via the the addition of superparamagnetic iron oxide (SPIO) particles. With the appropriate pulse sequence and imaging parameters, embedded SPIO particles enhance stent visibility under MRI after the stent is implanted within a body lumen, as well as permit a clinician to track the rate of stent degradation. As the biodegradable polymer degrades, the SPIO particles are released and either flow downstream or become phagocytosed by nearby macrophages. As the number of SPIO particles are reduced, the resulting MRI signal from the remaining stent structure will change. By quantifying the change in the MRI signal, the amount of biodegradable stent remaining can be deduced, allowing for accurate calculation of degradation rates.

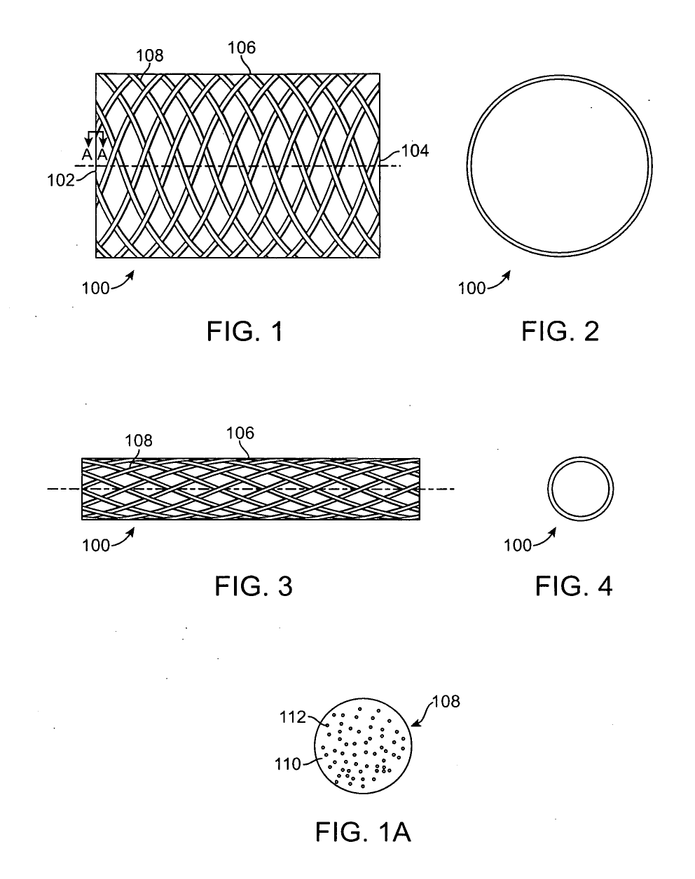

FIGS. 1-2 illustrate an endoluminal prosthesis, stent 100, in accordance with an embodiment hereof. Stent 100 includes a generally cylindrical hollow body portion 106 extending between a proximal end 102 and a distal end 104. Body portion 106 may have a generally tubular or cylindrical expandable structure that is configured to fit into a body lumen such as a blood vessel. As shown in FIG. 2, the cross-sectional shape of stent 100 may be circular. However, the cross-sectional shape may alternatively be ellipsoidal, rectangular, hexagonal rectangular, square, or other polygon. An outer diameter of body portion 106 may be approximately equal to or slightly larger than an inner diameter of a target body vessel and may be substantially constant along the length thereof. Body portion 106 is formed by one or more filaments 108. A typical stent device will comprise between 10 to 50 filaments, but more or less filaments may be used. In the embodiment depicted in FIG. 1, one set of filaments are in the form of helices which are axially displaced in relation to each other and have the center line of tubular body portion 106 as a common axis. Another set of filaments are also in the form of helices, which are axially displaced in relation to each other and also have the center line of tubular body portion 106 as a common axis; however, the second set of helices extend in the opposite direction relative to the first set of helices. The two sets of filaments cross each other at points in the manner shown in FIG. 1.

It will be appreciated by one of ordinary skill in the art that stent 100 of FIG. 1 is merely an exemplary stent and that stents of various forms can be used in accordance with various embodiments of the present invention. For example, filaments 108 may be employed in a wide variety of filament-based stents, particularly stents that contain coiled and/or braided, knitted, or otherwise woven filaments. Some suitable examples of stent structures are shown in U.S. Pat. No. 5,545,208 to Wolff et al. and U.S. patent application Ser. No. 11/909,297 to Cho et al, filed on Sep. 21, 2007, Attorney Docket No. P21949 US, each of which is incorporated by reference herein in its entirety.

Referring now to FIG. 1A, which is a cross-sectional view of filament 108 taken along line A-A of FIG. 1, filaments 108 of stent 100 are constructed out of a solid bioabsorbable/biodegradable polymeric material 110 having active agent particles 112 dispersed there through that are visible by magnetic resonance imaging (MRI). All filaments 108 within stent 100 may contain active agent particles 112, or alternatively only a portion of the filaments within the stent may contain active agent particles 112. Moreover, filaments 108 may differ from one another in other various ways, for example, with respect to size, shape, or polymer content. Filaments 108 may be formed from the same type of polymer or polymer blend, or they may be formed from differing polymers or polymer blends.

Polymeric material 110 is a bioabsorbable/biodegradable polymer that dissolves or breaks down within a vessel such that active agent particles 112 is released or emitted into the vessel lumen. The biodegradable polymer may include, for example, polyactic acid, polyglycolic acid, collagen, polycaprolactone, hylauric acid, co-polymers of these materials, as well as composites and combinations thereof. The bioabsorbable polymeric material may include polymers or copolymers such as polylactide [poly-L-lactide (PLLA), poly-D-lactide (PDLA)], polyglycolide, polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly(alpha-hydroxy acid) or two or more polymerizable monomers such as trimethylene carbonate, ε-caprolactone, polyethylene glycol, 4-tert-butyl caprolactone, N-acetyl caprolactone, poly(ethylene glycol) bis(carboxymethyl) ether. Each type of biodegradable polymer has a characteristic degradation rate in the body. Some materials are relatively fast-bioabsorbing materials (weeks to months) while others are relatively slow-bioabsorbing materials (months to years). The dissolution rate of filaments 108 may be tailored by controlling the type of bioabsorbable polymer, the thickness and/or density of the bioabsorbable polymer, and/or the nature of the bioabsorbable polymer. In addition, increasing thickness and/or density of a polymeric material will generally slow the dissolution rate of the filaments. Characteristics such as the chemical composition and molecular weight of the bioabsorbable polymer may also be selected in order to control the dissolution rate of the filaments.

The active agent particles 112 are visible to a physician viewing, for example, a magnetic resonance imaging (MRI) device while evaluating stent 100 after stent 100 has been implanted within the target body vessel. Active agent particles 112 are biocompatible superparamagnetic agents such as iron oxide, which is suitable as contrast media for MRI. The superparamagnetic agents cause magnetic field inhomogeneities, resulting in signal dephasing that manifests as local image distortion. The superparamagnetic agents shorten T2, the spin-spin time constant that characterizes the decay of the transverse magnetization, and T2*, the reduced T2 due to dephasing near inhomogeneities. By using a T2 or T2* weighted sequence, pronounced negative contrast, or signal drop-off, from the paramagnetic particles can be quantified.

The superparamagnetic agents may be composed of a magnetic iron oxide core surrounded by a coating that prevents aggregation and sedimentation of the particles in aqueous solutions, achieves high biological tolerance, and prevents toxic side effects. Suitable materials for the coating include but are not limited to dextran, carboxydextran, or siloxane with a polymer coating. The core size ranges from 2 nm to less than 10 nm, and the hydrodynamic diameter ranges from 20 nm to about 150 nm. Particles with a relatively larger size, such as greater than 50 nm hydrodynamic diameter, are called superparamagnetic iron oxide (SPIO) particles. Particles with a relatively smaller size, less than 50 nm hydrodynamic diameter, are called ultrasmall superparamagnetic iron oxide (USPIO) particles. For simplicity, unless otherwise directed, both SPIO particles and USPIO particles will be collectively referred to as superparamagnetic iron oxide (SPIO) particles herein.

SPIO particles have been used clinically for several years as contrast agents in MRI. As previously explained, under MRI, clusters of SPIO particles can be used to decrease the signal intensity on T2/T2* weighted images, via a shortening of T2/T2*, such that a signal drop-off or pronounced negative contrast occurs where the SPIO particles are present. Thus, stent 100 having SPIO particles embedded therein are detectable under MRI because the particles will cause inhomogeneities in the gradient magnetic field that are different from the surrounding tissue.

Due to their long half-life in blood, superparamagnetic iron oxide particles can be taken up, or phagocytosed, by macrophages in the whole body at a relatively high rate. As stent 100 biodegrades, active agent particles 112 are released and engulfed by macrophages or will otherwise flow downstream. The amount of active agent particles 112 embedded within stent 100 decreases, which lessens or reduces the signal drop-off caused by the superparamagnetic iron oxide particles. Stated another way, as the active agent particles 112 migrate from the degradation of stent 100, the signal drop-off or void caused by the SPIO particles will weaken and become more diffuse as the MRI signal of the surrounding tissue progressively intensifies. As will be explained in more detail below, this signal change allows for one to track the degradation of the stent in situ and accurately calculate the stent degradation rate.

In addition to active agent particles 112 that are visible by magnetic resonance imaging, filaments 108 may also include a therapeutic or other specific beneficial agent that is released into the vessel for treatment thereof as stent 100 biodegrades. A wide range of therapeutic agents can be used, with the pharmaceutically effective amount being readily determined by those of ordinary skill in the art and ultimately depending, for example, upon the condition to be treated, the nature of the therapeutic agent itself, the tissue into which the dosage form is introduced, and so forth. For example, the therapeutic agents may include one or more of the following: anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms. The therapeutic agents may be disposed within the filament or attached to the surface of the filament as a coating.

While FIGS. 1-2 illustrate stent 100 in an expanded state configured to contact a vessel wall, FIGS. 3-4 illustrate stent 100 in a contracted state that enables stent 100 to be delivered to the target site. In both the contracted and expanded states, stent 100 assumes a substantially tubular form. FIG. 2 shows the diameter of tubular body 106 in an expanded state, whereas FIG. 4 shows the diameter of tubular body 106 in a contracted state. The ends of tubular body 106 may be axially displaced relative to each other as the diameter of tubular body 106 is varied. For example, the length of tubular body 106 is greater in a contracted state shown in FIG. 3 than the length of tubular body 106 in an expanded state shown in FIG. 1.

Stent 100 can be expanded in a number of ways, including using a balloon or other stent-expanding device known in the art to exert outward radial expansion forces. When stent 100 is balloon expandable, stent 100 is formed in an expanded state, crimped onto a conventional balloon dilation catheter for delivery to a treatment site and expanded by the radial force of the balloon. Conventional balloon catheters that may be used in the present invention includes any type of catheter known in the art, including over-the-wire catheters, rapid-exchange catheters, core wire catheters, and any other appropriate balloon catheters. For example, conventional balloon catheters such as those shown or described in U.S. Pat. Nos. 6,736,827; 6,554,795; 6,500,147; and 5,458,639, which are incorporated by reference herein in their entirety, may be used within the stent delivery catheter of the present invention.

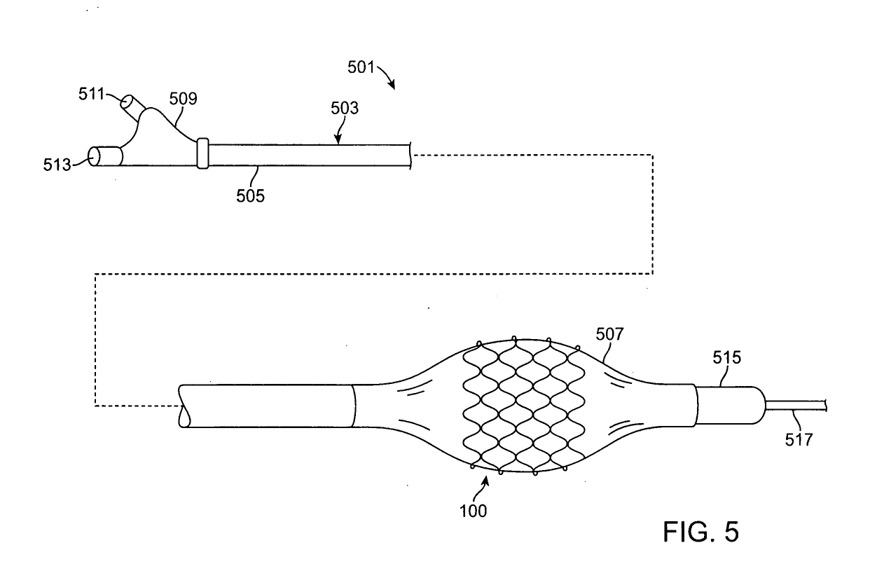

For example, FIG. 5 is an illustration of a stent delivery system 501 for tracking stent 100 to the target site in accordance with an embodiment of the present invention. Stent delivery system 501 includes a catheter 503 having a proximal shaft 505, a guidewire shaft 515, and a balloon 507. Proximal shaft 505 has a proximal end attached to a hub 509 and a distal end attached to a proximal end of balloon 507. Guidewire shaft 515 extends between hub 509 and a distal tip of catheter 503 through proximal shaft 505 and balloon 507. Hub 509 includes an inflation port 511 for coupling to a source of inflation fluid. Inflation port 511 fluidly communicates with balloon 507 via an inflation lumen (not shown) that extends through proximal shaft 505. In addition, hub 509 includes a guidewire port 513 that communicates with a guidewire lumen (not shown) of guidewire shaft 515 for receiving a guidewire 517 there through. As described herein, guidewire shaft 515 extends the entire length of catheter 503 in an over-the-wire configuration. However, as would be understood by one of ordinary skill in the art, guidewire shaft 515 may alternately extend only within the distal portion of catheter 503 in a rapid-exchange configuration. A stent 100 having biodegradable filaments 108 with active agent particles 112 dispersed there through that are visible by magnetic resonance imaging (MRI) formed in accordance with an embodiment described herein is positioned over balloon 507. If desired, a sheath (not shown) may be provided to surround stent 100 to facilitate tracking of the stent delivery system 501 over guidewire 517 through the vasculature to a site of a stenotic lesion.

Deployment of balloon expandable stent 100 is accomplished by tracking catheter 503 through the vascular system of the patient until stent 100 is located within a target vessel. The treatment site may include target tissue, for example, a lesion which may include plaque obstructing the flow of blood through the target vessel. Once positioned, a source of inflation fluid is connected to inflation port 511 of hub 509 so that balloon 507 may be inflated to expand stent 100 as is known to one of ordinary skill in the art. Balloon 507 of catheter 503 is inflated to an extent such that stent 100 is expanded or deployed against the vascular wall of the target vessel to maintain the opening. Stent deployment can be performed following treatments such as angioplasty, or during initial balloon dilation of the treatment site, which is referred to as primary stenting.

In another embodiment hereof, stent 100 may be self-expanding due to the inherent resiliency of particular biodegradable materials such as, for example, poly-L-lactide, poly-D-lactide, polyglycolide, such that filaments 108 return to an expanded state when released from a compressed state. For instance, stent 100 may be introduced into a body lumen inside a sleeve or sheath that maintains stent 100 in a compressed, reduced size. When the stent is positioned within the body lumen at the target site such as, for example, an occlusion, the sleeve is proximally withdrawn in the direction towards the operator, enabling stent 100 to radially expand by its own internal restoring forces and engage the occlusion as well as the adjacent healthy wall of the lumen. After deployment, stent 100 expands and pushes the lumen walls outward to open the lumen.

As previously mentioned, embedding SPIO particles in biodegradable polymeric stents will not only enhance visibility of the stent under MRI, but it will also allow for tracking/monitoring of stent degradation. As the stent degrades, the SPIO particles will either be phagocytosed by nearby macrophages, becoming markers for inflammation nearby and distal to the implant site, or will flow downstream. The amount of SPIO particles embedded within the stent decreases, thus causing the MRI signal from the remaining stent structure to change. For example, the signal change can be calculated by quantifying the pixel intensity in the vicinity of the SPIO particles before and after degradation, and then performing a subtraction to determine the difference in signal. By quantifying the change in the MRI signal, the amount of biodegradable stent remaining can be deduced, allowing for accurate calculation of the stent degradation rate.

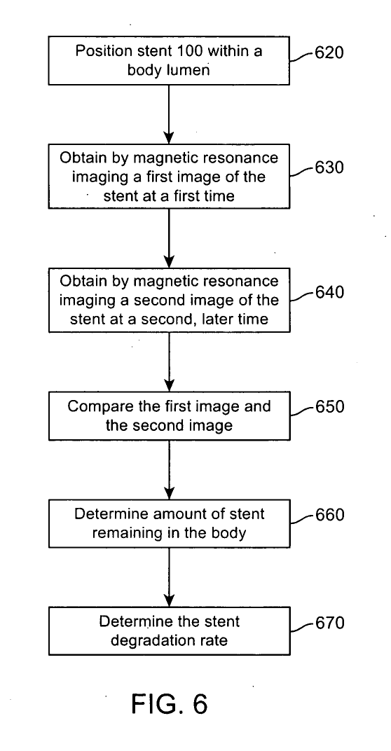

More particularly with reference to FIG. 6, in order to non-invasively track the degradation of a biodegradable polymeric stent, stent 100 is positioned within a body lumen of a patient as shown in step 620. As described above, stent 100 includes superparamagnetic iron oxide particles embedded into the biodegradable polymeric material. A first magnetic resonance image (MRI) of stent 100 is obtained immediately after the stent is positioned within a body lumen at step 630. The first MRI image is obtained using an appropriate pulse sequence and signal parameters, discussed in more detail below, such that the embedded SPIO particles within stent 100 are detectable under MRI. At step 640, a second magnetic resonance image (MRI) of stent 100 is obtained during follow-up procedure/appointment, or at a time after the first image is obtained. In one embodiment, the second image may be obtained between 1-12 months after the stent is positioned within a body lumen. However, the second image may be obtained at any time along the degradation cycle. The second image is obtained with the same pulse sequence and signal parameters as utilized to obtain the first signal so that a direct comparison between the two images may be performed. In step 650, the first and second images are compared to each other in order to determine the amount of stent remaining in the body lumen. Under MRI, with T2* weighting, a signal drop-off or void occurs where SPIO particles are present. Thus, as stent 100 degrades, the amount of SPIO particles embedded within stent 100 decreases, hence lessening or reducing the signal drop-off caused by the SPIO particles as the MRI signal of the surrounding tissue progressively returns. The signal change between the first and second images is quantified (such as, for example, in the form of a percentage or fraction) by comparing the amount of signal drop-off in the first image to the amount of signal drop-off in the second image. Such quantification allows the operator to track or monitor the degradation of the stent in situ and determine the amount of stent material remaining in the body at step 660. Further, once the amount of stent remaining in the body has been determined, the degradation rate of stent 100 may be determined at step 670. The stent degradation rate is calculated via the ratio of the amount of stent that has degraded and the time period that has passed between the time the first image was obtained and the time the second image was obtained.

The first image of stent 100, which is normally obtained immediately after stent 100 is positioned within a body lumen, is thus used as a baseline for comparison during follow-up procedures/appointments. Since stent 100 will include the embedded SPIO particles, the first image will include a signal drop-off or void and may be considered one end of the signal spectrum. In addition, another, or third, magnetic resonance image (MRI) of an area upstream of stent 100 may be taken upon implantation using the same pulse sequence and signal parameters as utilized to obtain the first signal. This upstream image may also be used as a baseline for comparison during follow-up procedures/appointments. Since the area upstream of stent 100 will not include any SPIO particles, the upstream image will include a MRI signal of tissue upstream of stent 100 and will not include a signal drop-off or void, and thus may be considered the opposite end of the signal spectrum. When a second or follow-up image of the stent is obtained during a follow-up procedure/appointment using the same pulse sequence and signal parameters as utilized to obtain the first signal, which takes place after a portion of stent 100 has degraded, the signal drop-off or void of the second image will fall somewhere along the signal spectrum between the baselines or ends represented by the first, baseline image of the stent and the third, upstream image of the body lumen.

USPIO particles having a relatively smaller particle size (less than 50 nm hydrodynamic diameter) have a longer intravascular half-life than SPIO particles having a relatively larger particle size (greater than 50 nm hydrodynamic diameter), and therefore would be more likely to be engulfed by macrophages in the vicinity of the stent implant site. Thus, the relatively smaller USPIO particles may be useful for imaging any inflammatory response near the implant. However, when calculation of the rate of degradation is of primary interest, the relatively larger SPIO particles (greater than 50 nm hydrodynamic diameter) may be more useful for enhancing the signal difference between the MRI images, as the larger particles will clear from the implant site more quickly and will not prolong the distortion effects post stent degradation.

In order to obtain the MRI images such that the embedded SPIO particles within stent 100 result in a negative contrast and are visible/detectable under MRI, a T2 or T2* gradient echo pulse sequence based on a flip angle between 0° and 90° may be used. In addition, other pulse sequences may be utilized to produce MRI images of a stent having SPIO particles embedded therein, including but not limited to fast spin echo and fast gradient-recalled echo sequences. The pulse sequence may have two parameters to control weighting, echo time (TE) which is the time between the RF pulse and MR signal sampling and repetition time (TR) which is the time between two excitation pulses. For imaging SPIO particles, the pulse sequence is preferably obtained with T2/T2* weighting techniques (a long TR and long TE sequence) because SPIO particles have a predominant T2/T2* shortening effect and produce a drop in signal namely in T2/T2* weighting. Other factors that are under operator control and may be tailored to produce optimal MRI images include sequence parameter times, matrix size, slice thickness and gap between slices, field-of-view (FOV), number of excitations, orientation of imaging plane, and type of receiver coil.

Alternatively, other pulse sequences or T1-weighting may be used to enhance the signal generated by the SPIO particles, resulting in positive contrast. Using a modified spin echo pulse sequence, a positive contrast for iron oxide particles may be produced. The methodology to create such a positive contrast employs spectrally selective radiofrequency pulses to excite and refocus the off-resonance water surrounding the iron-containing cells while suppressing the on-resonance signal. With such positive contrast sequences, clusters of SPIO particles increase the signal intensity such that a more intense or pronounced positive contrast occurs where the SPIO particles are present. Thus, the signal from the SPIO particles embedded in the stent will decrease as the stent degrades and the SPIO particles are released downstream.

Stent 100 may be manufactured by first preparing a SPIO-polymer composite. The SPIO-polymer composite may be formed by dissolving a biodegradable polymer in an appropriate solvent and then introducing SPIO particles. Suitable solvents that may be utilized include but are not limited to diluted acidic solutions, diluted basic solutions, neutral solutions, alcohols and mixtures thereof. The solvent is evaporated to result in a solid composite including the biodegradable polymeric material and superparamagnetic iron oxide particles. The solid composite is then extruded into filaments 108. Once filaments 108 are provided, the tubular stent body can be formed using various wire forming techniques known in the stent art, such as coiling techniques or weaving techniques (e.g., braiding or knitting).

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of non-invasively tracking the degradation of a biodegradable stent implanted in a body lumen, the method comprising the steps of:
providing a stent of a biodegradable polymeric material with superparamagnetic iron oxide particles embedded therein;
positioning the stent within the body lumen;
obtaining by magnetic resonance imaging a first image of the stent at a first time;
obtaining by magnetic resonance imaging a second image of the stent at a second time, wherein the second time occurs after the first time; and
comparing the first image and the second image to determine the amount of stent remaining in the body lumen.

2. The method of claim 1, further comprising:
quantifying the amount of stent remaining in the body lumen and the amount of stent that has degraded over the time period that has passed between the first time and the second time; and
calculating the degradation rate of the stent.

3. The method of claim 1, wherein the first image includes a first signal having a first amount of signal intensity and the second image includes a second signal having a second amount of signal intensity, and the step of comparing the first image and the second image includes quantifying the change between the first amount of signal intensity and the second amount of signal intensity.

4. The method of claim 1, wherein the stent has a tubular body defined by a plurality of extruded braided filaments.

5. The method of claim 1, wherein the first time is when the stent is initially positioned within the body lumen.

6. The method of claim 1, wherein the second time is during a time period of 1-12 months after the stent is initially positioned within the body lumen.

7. The method of claim 1, further comprising the step of:
obtaining by magnetic resonance imaging a third image of an area of the body lumen upstream of the stent.

8. The method of claim 1, wherein the biodegradable polymeric material is selected from the group consisting of polyactic acid, polyglycolic acid, collagen, polycaprolactone, hylauric acid, polylactide, polyglycolide, polydioxanone, polycaprolactone, polygluconate, polylactic acid-polyethylene oxide copolymers, modified cellulose, collagen, poly(hydroxybutyrate), polyanhydride, polyphosphoester, poly(amino acids), and poly(alpha-hydroxy acid).

9. The method of claim 1, wherein the superparamagnetic iron oxide particles have a particle size between 10 nm and 150 nm.

10. The method of claim 1, wherein the superparamagnetic iron oxide particles have a particle size greater than 50 nm.

11. The method of claim 1, wherein the steps of obtaining a first image and obtaining a second image are performed using the same pulse sequence and imaging parameters.

12. The method of claim 11, wherein the pulse sequence is a gradient echo pulse sequence.

13. The method of claim 11, wherein the pulse sequence is T2 or T2* weighted to achieve negative contrast of the superparamagnetic iron oxide particles.

14. The method of claim 11, wherein the pulse sequence is T1 weighted to achieve positive contrast of the superparamagnetic iron oxide particles.

15. A method of manufacturing a stent imageable under magnetic resonance imaging when placed in a body lumen of a patient, the method comprising the steps of:
dissolving a biodegradable polymeric material in a solvent;
introducing superparamagnetic iron oxide particles into the solvent and biodegradable polymeric material mixture;
evaporating the solvent to result in a solid composite including the biodegradable polymeric material and superparamagnetic iron oxide particles;
extruding the solid composite into fibers; and
braiding the fibers to generate a tubular stent body.

16. The method of claim 15, wherein the biodegradable polymeric material is selected from the group consisting of polyactic acid, polyglycolic acid, collagen, polycaprolactone, hylauric acid, polylactide, polyglycolide, polydioxanone, polycaprolactone, polygluconate, polylactic acid-polyethylene oxide copolymers, modified cellulose, collagen, poly(hydroxybutyrate), polyanhydride, polyphosphoester, poly(amino acids), and poly(alpha-hydroxy acid).

17. The method of claim 15, wherein the superparamagnetic iron oxide particles have a particle size between 10 nm and 150 nm.

18. The method of claim 15, wherein the superparamagnetic iron oxide particles have a particle size greater than 50 nm.

19. The method of claim 15, wherein the solvent is selected from the group consisting of diluted acidic solutions, diluted basic solutions, neutral solutions, alcohols and mixtures thereof.
